(19) United States
(12) Patent Application Publication
(10) Pub. No.: US 2023/0174942 A1
(43) Pub. Date: Jun. 8, 2023

(54) NOVEL FABRICATION OF CORONARY BASED DECELLULARIZED HEART FLAPS TO TREAT ANEURYSM FOLLOWING MYOCARDIAL INFARCTION

(71) Applicant: AtRoo, Inc., Watertown, MA (US)

(72) Inventors: Aram AKBARZADEH, Cambridge, MA (US); David Elmaleh, Newton, MA (US); Abdol-mohammad Kajbafzadeh, Tehran (IR)

(73) Assignee: AtRoo, Inc., Watertown, MA (US)

(21) Appl. No.: 17/862,803

(22) Filed: Jul. 12, 2022

Related U.S. Application Data
(60) Provisional application No. 63/220,814, filed on Jul. 12, 2021.

(51) Int. Cl. C12N 5/077; C12M 1/00; A61P 9/00

(52) U.S. Cl. CPC C12N 5/0657; C12M 29/10; A61P 9/00

(57) ABSTRACT

Some aspects of this disclosure provide methods of producing decellularized and recellularized cardiac tissues engineering methods of treating heart disease, including heart aneurysm and myocardial infarction. Cardiac tissue engineering of compositing and constructing of a recellularized coronary-based right heart flap or cardiac flap scaffold with its cellular components are also disclosed.

### CROSS REFERENCE TO RELATED APPLICATIONS

This Application claims the benefit under 35 U.S.C. § 119(e) of the priority of U.S. Provisional Patent Application Ser. No. 63/220,814, filed Jul. 12, 2021, the entirety of which is hereby incorporated by reference for all purposes. The following related applications and materials are incorporated herein, in their entireties, for all purposes: U.S. Provisional Patent Application Ser. No. 63/220,814.

### FIELD

Some aspects of this disclosure provide methods of producing decellularized and recellularized cardiac tissues engineering methods of treating heart disease, including heart aneurysm and myocardial infarction. Cardiac tissue engineering of, compositing, and constructing of a recellularized coronary-based right heart flap or cardiac flap scaffold with its cellular components are also disclosed.

### BACKGROUND

Cardiovascular diseases have been considered as the leading cause of mortality in the world, accounting for 17.3 million deaths annually (Nees S N, Chung W K. Genetic Basis of Human Congenital Heart Disease. Cold Spring Harb Perspect Biol. 2019:a036749). Myocardial infarction (MI) results in replacement of non-functioning fibrotic tissue and loss of functioning cardiomyocytes. Left ventricular aneurysms (LVAs) and pseudoaneurysms are two grave complications of MI that if left undetected, it can lead to death or serious morbidity. A ventricular free wall ruptures along with the pericardium and the absence of myocardial tissues in its wall, are catastrophic complications occurring in 4% of patients after MI (Sheikh W R, Sehgal P, Verma A, Haldar M. Jaiswal S. Left ventricular pseudoaneurysm post myocardial infarction. Int J Crit illn Inj Sci. 2019; 9(1):43).

The most commonly considered surgical interventions for LVAs are coronary artery bypass graft surgery, which can be combined if needed with surgical ventricular reconstruction (SVR) or ventricular reconstructive surgery. The main goal in treating LVAs is to correct the size and geometry of the left ventricle (LV) in order to reduce wall tension and paradox movement and to improve systolic function. Intracavitary thrombi are removed, and coronary artery bypass grafting (CABG) is usually performed, typically with synthetic (Dacron) or bovine pericardial patches.

Ventricular reconstructive surgery is a very high-risk surgical option for treating LVAs. (Castelvecchio, Serenella, et al. “Surgical ventricular reconstruction for ischaemic heart failure: state of the art.” European Heart Journal Supplements 18.suppl_E (2016): E8-E14.). Another limitation of SVR is that it fails to maintain the improvements over a long-term period because of the recurrent dilatation of the ventricle. This is partly because of the shortcomings in the repairing patches that often accompany the surgery. The materials typically used for these cardiac patches are typically stiff and synthetic, which render the patch and the adjacent regions scarred and nonelastic. This results in chronic stresses and contributes to ventricular redilatation and dysfunction. (Qu. Hui, et al. “Improved left ventricular aneurysm repair with cell- and cytokine-seeded collagen patches.” Stem cells international 2018 (2018)

The current synthetic or bovine grafts available for treatment of left ventricular aneurysms are prone to infection, thrombosis, and subsequent dehiscence of the anastomosis and pseudoaneurysm (Kurobe H, Maxfield M W, Breuer C K. Shinoka T. Concise review: Tissue-engineered vascular grafts for cardiac surgery: Past, present, and future. Stem Cells Transl Med. 2012; 1(7):566-71). The application of these grafts is far from ideal, especially in the pediatric population, as it may create a high risk of re-infection and it also requires multiple operations as a child grows. The currently available synthetic grafts are incapable of connecting to the circulatory system to receive nutrition and oxygen. The aneurismal thinning of the ventricular wall should be conferred with structural support using natural thin decellularized slices, as patch-based therapies in combination with neonatal cardiomyocytes in order to provide an efficient environment for scar replacement therapy. Many efforts have been made in the development of scaffolds and optimization of the microenvironment for management of aneurysm following MI, however, cell source still remains one of the bottleneck troubles. Although mature vascular cells isolated from the donor and adult stem cells have been used to regenerate heart tissue, these cells may not be sufficient in number with limited proliferation potentials during in vitro expansion. Furthermore, proliferation and differentiation abilities of adult stem cells reduce with increasing donor age, which limits their realistic usage in elderly patients (Wang Y, Hu J, Jiao J, Liu Z, Zhou Z, Zhao C et al. Engineering vascular tissue with functional smooth muscle cells derived from human iPS cells and nanofibrous scaffolds. Biomater. 2014; 35(32):8960-9). The efficacy of decellularized extracellular matrix (ECM) has been also verified for further heart regeneration therapies after MI (Akhyari, Payam, et al. “The quest for an optimized protocol for whole-heart decellularization: a comparison of three popular and a novel decellularization technique and their diverse effects on crucial extracellular matrix qualities.” Tissue Engineering Part C: Methods 17.9 (2011): 915-926). The quest for an optimized protocol for whole-heart decellularization: a comparison of three popular and a novel decellularization technique and their diverse effects on crucial extracellular matrix qualities. Tissue Eng C. 2011; 17(9):915-26).

The preservation of crucial ECM elements and the 3D architecture of the cardiac tissue are also among the advantages of the application of heart decellularized ECM, for further cell support and differentiation (Jallerat Q, Feinberg A W. Extracellular Matrix Structure and Composition in the Early Four-Chambered Embryonic Heart. Cells. 2020; 9(2):285). Considering the complex structure of the heart tissue, providing an appropriate scaffold would result in efficient further recellularization. The capability of green fluorescent protein (“GFP”) rat neonatal cardiac cells in constructing cardiac tissue on pre-designed three-dimensional (3D) biodegradable sheep heart scaffold are tested in the present Example.

The use of decellularized patch dissected from the atrium and ventricle without coronary arteries to treat LVAs has not been successful to date. Previous studies and research investigated the use of rat heart atrial and ventricular decellularized cardiac ECM patches with a thickness of about 300 μm to about 800 μm; however, scaffolds thicker than a few hundred microns can cause significant cell death, especially in the central core of the scaffold. This is because in native tissue, most cells can survive within 100 μm to 200 μm from the nearest capillary for sufficient diffusion of oxygen and nutrients. These thicker scaffolds, such as the rat heart atrial and ventricular ECM patches, put extra weight on the pumping heart which can increase the afterload on the injured left ventricle and accelerate the negative remodeling process.

Synthetic biodegradable polymers have also been investigated for potential treatment of LVAs. However, polymers such as polycaprolactone (PCL) exhibit a long degradation time, which is an obstacle for use in cardiac tissue engineering. (Nasr, Saeed Mohammadi, et al. “Biodegradable nanopolymers in cardiac tissue engineering: from concept towards nanomedicine.” International Journal of Nanomedicine 15 (2020): 4205) As such, there is a need for a suitable cardiac graft that will repair aneurysms following myocardial infarction.

The present invention overcomes the problems of the current strategies by employing the right heart chamber, specifically the right atrium and right ventricle, which are much thinner and less dense than the left counterpart. The right chamber is highly superior to the left for cardiac patch tissue engineering. By grafted coronary anastomosis to the aorta, the patch of the current invention is grafted to the heart, the in vitro seeded cells in the patch receive nutrient and oxygen, and new circulating bone marrow stem cells come to the patch and differentiate to several cardiac homing cell, resulting in normal homogenous cardiac muscles. These new cells integrate with the previous in vitro seeded cells including; endothelial, fibroblasts, cardiomyocytes etc.

The heart patches of the present invention protect whole infarcted heart by neo-vascularization and supportive cardiac pump with very young non atherosclerotic arteries. In contrast to synthetic material such as Dacron and polytetrafluoroethylene or bovine pericardial patch for aneurism treatment, the patches of the present invention are fully vascularized and recellularized not only due to in vitro cell seeding but following DCABG; by grafted coronary anastomosis to the aorta the circulating bone marrow stem cells creating angioneogenesis and finally revascularization in order to create a homogenous patch.

The present invention overcomes the problems and disadvantages associated with current strategies for the treatment of heart disease, including repairing aneurysm following myocardial infarction. The present decellularized/recellularized cardiac heart flaps disclosed herein overcomes the issues with synthetic heart patches which develop biofilms and lose flexibility or elasticity over time. The lack of blood flow through the synthetic heart patches leads to the rupturing of a pre-existing or new aneurysm. The present invention can connect to the circulatory system, which in turn provides nutrition and oxygen to the implanted heart flap. This is achievable in part because the heart flap contains a right coronary. Another improvement of the present invention over the existing strategies is the ability to improve the distribution and effectiveness of prescribed cardiac medicines. This is due in part because cardiac medicines are effective primarily on the parts of the heart that are alive and not on dead fibrotic cardiac tissue. If cardiac medicines prescribed for a normal healthy heart are taken by a subject suffering from a LVA, there is a potential for more aneurism ballooning and the possibility of more rupturing. The heart flaps of the present invention will prevent left ventricular ballooning, increase the cardiac output, and finally the heart ejection fraction which reduces the potential for heart failure with a better quality of life.

### SUMMARY

In some embodiments, the present teachings provide a method of producing a decellularized tissue matrix or decellularized myocardial matrix comprising. (a) providing a tissue sample or myocardial sample, and (b) perfusing said tissue sample or organ sample, thereby producing a decellularized tissue matrix or decellularized organ matrix.

In some embodiments, the present teachings provide a method of producing a decellularized cardiac tissue matrix or decellularized heart matrix comprising: (a) providing a cardiac tissue sample or right heart sample, and (b) perfusing said tissue sample or right heart, thereby producing a decellularized tissue matrix or decellularized organ matrix, wherein said step (a) of providing a tissue sample or organ sample comprises dissecting a heart.

In some embodiments, the present teachings provide a method of producing a decellularized cardiac tissue matrix or decellularized right heart matrix.

In some embodiments, the present teachings provide a method of producing a decellularized mammalian cardiac tissue matrix comprising: (a) providing the right heart flap or the right heart of a mammal by dissection, (b) cannulating the right coronary artery (“RCA)” (c) perfusing said right heart flap or right heart with distilled water, (d) perfusing said right heart flap or right heart with 1% SDS, and (e) rising said right heart flap or right heart with phosphate buffered saline (PBS), containing 100 U/ml penicillin. 100 U/ml streptomycin, thereby producing a decellularized cardiac tissue matrix.

In some embodiments, the present teachings provide a method of producing a decellularized mammalian cardiac tissue matrix comprising: (a) cannulating a mammalian right heart flap, mammalian right heart, or mammalian right coronary artery, (b) perfusing said right heart flap or right heart with distilled water, (c) perfusing said right heart flap or right heart with 1% SDS, and (d) rising said right heart flap or right heart with phosphate buffered saline (PBS), containing 100 U/ml penicillin. 100 U/ml streptomycin, thereby producing a decellularized cardiac tissue matrix.

In some embodiments, the present teachings provide a method of producing a recellularized cardiac flap by repopulating a decellularized cardiac flaps with GFP positive neonatal cardiac cells, the method comprising: (a) connecting the decellularized cardiac flaps to a closed-system bioreactor that is placed in a cell culture incubator with about 5% CO2 at about 37° C., (b) circulating the medium with 10 mL/min via the right coronary of said cardiac flap for about 24 h, (c) removing the decellularized cardiac flaps, (d) perfusing the flaps with neonatal cardiac cells via the right coronary arteries of the cardiac flaps, (e) injecting 5 million GFP⁺ rat neonatal cardiac cells into five different areas of the myocardium on day 5 and day 10 thereby producing an appropriate recellularized cardiac flap ready for cardiac treatment by implantation.

In some embodiments, the present teachings provide a method of producing a recellularized cardiac flap by repopulating a decellularized cardiac flaps with GFP positive rat neonatal cardiac cells, the method comprising: (a) perfusing the flaps in a medium with predetermined quantity of ten million GFP+ rat neonatal cardiac cells upon, (b) injecting 5 million GFP+ rat neonatal cardiac cells into five different areas of the myocardium on day 1 and day 5 repeated predetermined days, thereby producing the recellularized cardiac flap ready for implantation treatment.

In some embodiments, the present teachings provide a recellularized cardiac flap produced by any examples or methods disclosed herein.

In some embodiments, the present teachings provide method of treating or repairing a heart aneurysm in a subject requiring cardiac patch or flap grafting onto the heart, by replacing a damaged part of the heart with a recellularized cardiac patch or flap, or by implanting a recellularized cardiac patch or flap on a ventricular surface.

In one embodiment, the present teachings provide a method of preventing a left ventricular free wall rupture following acute myocardial infarction in a subject by grafting a recellularized cardiac patch or flap onto the heart, by replacing a damaged part of the heart with a the recellularized cardiac patch or flap, or by implanting a recellularized cardiac patch or flap on a ventricular aneurysm surface without removing the scarred, dead area of heart tissue or the aneurysm.

In some embodiments, the present teachings provide a method of treating a ventricular aneurysm by grafting a recellularized cardiac patch or flap onto the heart. Replacing a damaged part of the heart with a the recellularized cardiac patch or flap, or by implanting a recellularized cardiac patch to restore cardiac muscular activity.

In one embodiment, the present teachings provide a method of treating a myocardial infarction in a subject by grafting a recellularized cardiac patch or flap onto the heart, by replacing a damaged part of the heart with the recellularized cardiac patch or flap, or by implanting a recellularized cardiac patch or flap in ventricular aneurysm surface.

In some embodiments, the present teachings provide a method of repairing a heart aneurysm in a subject following a myocardial infarction by grafting a recellularized cardiac patch or flap onto the heart, by replacing a damaged part of the heart with the recellularized cardiac patch or flap, or by implanting a recellularized cardiac patch or flap on a ventricular surface.

In some embodiments, the present teachings provide a method of treating a heart aneurysm in a subject following a myocardial infarction by grafting a recellularized cardiac patch or flap onto the heart, by replacing a damaged part of the heart with the recellularized cardiac patch or flap, or by implanting a recellularized cardiac patch or flap on a ventricular aneurysm surface.

Other advantages, features, and characteristics of the present invention will become more apparent upon consideration of the following detailed description and claims.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIGS. 1A-1D shows the progressive changes from the decellularization process of the heart muscle. FIG. 1D shows the flap which was decellularized by perfusing the distilled water for 5 hours, followed by perfusion of 1% SDS for 216 hours with a flow rate of approximately 40 ml/min, then rinsing with phosphate buffered saline (PBS) for another 240 hours.

FIGS. 2A-2F: FIGS. 2A-2B show H&E staining for natural sheep heart (FIG. 2A) and decellularized sheep heart (FIG. 2B). FIGS. 2C-2D show Trichrome staining at for natural sheep heart (FIG. 2C) and decellularized sheep heart (FIG. 2D). FIGS. 2E-2F show DAPI staining at for native sheep heart (FIG. 2E) and decellularized sheep heart (FIG. 2F).

FIG. 2G shows a chart of the DNA contents of the decellularized scaffold compared with that of the native ones.

FIG. 2H shows a chart comparing the average amounts of soluble collagen between the native and decellularized right ventricle.

FIG. 2I shows a chart comparing the average amounts of sulfated glycosaminoglycans (sGAGs) between the native and decellularized right ventricle.

FIGS. 3A-3F show scanning electron microscopy of native a (FIGS. 3A-3C) and decellularized right ventricle (FIG. 3D-3F).

FIGS. 3G-3I show the results of the tensile test and the mechanical properties of native and decellularized right ventricle and right atrium. Stress-strain curves: NRA: Normal right atrium, DRA: Decellularized right atrium, NRV: Normal right ventricle. DRV: Decellularized right ventricle.

FIG. 4 shows the preservation of the right coronary artery and vascular pedicle of the right heart after the decellularization process in the analysis of CTA images.

FIG. 5 shows the GFP+ rat cardiac cells proliferation profile by MTT assay. Cytotoxicity of the heart scaffold defined as a comparison of the viability of cells grown in wells lacking decellularized myocardial slices and the cells grown in wells in the presence of decellularized myocardial slices in the culture medium which shows no significant differences in optic density values among the two groups (p>0.05). Data are shown as means±SD. N=10 for each group after 48 h.

FIGS. 6A-6B show rat GPF positive cardiac cells (FIG. 6A) and DAPI staining of recellularized right heart scaffold after 20 days of recellularization (FIG. 6B).

FIGS. 7A-7E depict histological evaluations of the in-vitro recellularized right heart scaffold with H&E staining (FIG. 7A), and IHC staining of CD34 (FIG. 7B), Desmin (FIG. 7C), α-SMA (FIG. 7D), and Vimentin (FIG. 7E).

### DETAILED DESCRIPTION

In order to have a clearer and more consistent understanding of the report and the claims, including the scope given to said terms, the following definitions are provided.

Terms and phrases used herein are defined as set forth below unless otherwise specified. Throughout this description, the singular forms “a,” “an,” and “the” include plural reference unless the context clearly dictates otherwise. Thus, for example, a reference to “a cell” includes a plurality of such cells, and a reference to “an antibiotic” is a reference to one or more antibiotics and equivalents thereof known to those skilled in the art, and so forth.

For the purposes of clarity, the following abbreviations and terms are defined herein: Coronary artery bypass graft (CABG); 4′,6-diamidino-2-phenylindole (DAPI); Green fluorescent protein (GFP): Extracellular matrix (ECM); computed tomography angiography (CTA); Myocardial infarction (MI); Aortic valve conduit (AVC): Mesenchymal stem cell (MSC); Sodium dodecyl sulfate (SDS): Phosphate buffered saline (PBS); Scanning electron microscopy (SEM); and Immunohistochemistry (IHC).

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

The term “extracellular matrix”, abbreviated “ECM”, refers to the complex structural material that is produced by cells in mammalian tissues, particularly cells of connective tissue, for instance such cells as fibroblasts, osteoblasts, chondrocytes, epithelial cells, smooth muscle cells, adipocytes, and mesenchymal cells, and which material in vivo surrounds and supports those cells. Typically, the ECM is composed of fibers embedded in what is commonly referred to as “ground substance”. The fibers are composed of structural proteins, generally collagen and/or elastin. In aspects of the present invention, the fibers of the matrix are preferably collagen.

The term “matrix” refers to the structural component of the cell microenvironment. Matrix is also commonly referred to as “extracellular matrix” or “ECM.”

The terms “cardiac myocyte” and “cardiomyocyte” are used interchangeably throughout. The terms “GFP⁺ rat neonatal cardiac cells” and “GFP-positive rat neonatal cardiac cells” are used interchangeably throughout the present disclosure.

Biological tissues and organs can be decellularized using any number of known methods. For example, a biological tissue or organ can be decellularized using perfusion methods. See, for example, WO 2007/025233 and Ott et al. (2008, Nat. Med., 14:213-21) for descriptions of perfusion-based decellularization methods. Perfusion methods of decellularization have been shown to produce intact matrix for recellularization. See, for example, WO 2007/025233; Ott et al. (2008, Nat. Med., 14:213-21); Uygun et al., 2010, Nat. Med., 16(7):814-20; Petersen et al., 2010, Science, e-pub June; and Ott et al., 2010, Nat. Med., e-pub July.

In one embodiment, disclosed herein is a method of producing a decellularized cardiac tissue or heart organ matrix. In another embodiment, disclosed herein is a method of producing a decellularized cardiac tissue matrix or decellularized heart organ matrix.

In one embodiment, the decellularized cardiac tissue matrix or decellularized heart organ matrix disclosed herein are produced by perfusion-based methods. In another embodiment, the decellularized cardiac tissue matrix or decellularized heart organ matrix disclosed herein is a perfusion-based decellularized heart flap. In another embodiment, the decellularized cardiac tissue matrix or decellularized heart organ matrix disclosed herein is a perfusion-based decellularized right heart flap.

In one embodiment, the decellularized cardiac tissue matrix or decellularized heart organ matrix disclosed herein are produced by immersion in a decellularization solution.

In one embodiment, disclosed herein is a method of producing a decellularized cardiac tissue or heart organ matrix comprising: (a) providing a mammalian cardiac tissue or heart organ sample, (b) perfusing said cardiac tissue or heart organ sample, thereby producing a decellularized cardiac tissue or heart organ matrix. In another embodiment, disclosed herein is a method of producing a decellularized cardiac tissue matrix or decellularized heart organ matrix comprising: (a) providing a mammalian cardiac tissue or heart organ sample, (b) perfusing or immersing said cardiac tissue sample or heart organ sample or organ sample, thereby producing a decellularized tissue matrix or decellularized organ matrix.

In one embodiment, disclosed herein is a method example of producing a decellularized, cardiac ovine tissue, cardiac or sheep tissue, ovine heart, or sheep heart organ matrix comprising: (a) providing an ovine or sheep cardiac tissue or heart organ sample, (b) perfusing said cardiac ovine or sheep tissue or heart sample, thereby producing a decellularized, ovine or sheep cardiac tissue or organ matrix.

In one embodiment, disclosed herein is a method of producing a decellularized, porcine tissue or organ matrix comprising: (a) providing a porcine tissue or organ sample, (b) perfusing said porcine tissue or organ sample, thereby producing a decellularized, porcine tissue or organ matrix.

In one embodiment, disclosed herein is a method of producing a decellularized, human tissue or organ matrix comprising: (a) providing a human tissue or organ sample, (b) perfusing said human tissue or organ sample, thereby producing a decellularized, human tissue or organ matrix.

In one embodiment, disclosed herein is a method of producing a decellularized mammalian cardiac tissue matrix or a decellularized mammalian heart organ matrix comprising: (a) implanting a mammalian right heart flap or mammalian right heart, (b) perfusing said right heart flap or right heart with distilled water, (c) perfusing said right heart flap or right heart with 1% SDS, and (d) rising said right heart flap or right heart, thereby producing a recellularized mammalian cardiac tissue matrix or a recellularized mammalian heart organ matrix.

In one embodiment, disclosed herein is a method of producing a decellularized mammalian cardiac tissue matrix or a decellularized mammalian heart organ matrix comprising: (a) providing the right heart flap or the right heart of a mammal by dissection, (b) cannulating the right coronary artery, (c) perfusing said right heart flap or right heart with distilled water, (d) perfusing said right heart flap or right heart with 1% SDS, and (e) rising said right heart flap or right heart with phosphate buffered saline (PBS), containing 100 U/ml penicillin. 100 U/ml streptomycin, thereby producing a decellularized cardiac tissue matrix or a decellularized mammalian heart organ ready for treatment.

In one embodiment, the decellularized matrices disclosed herein are produced ex vivo. In one embodiment, the decellularized matrices disclosed herein are produced in vitro.

In one embodiment, disclosed herein are materials and methods for decellularizing tissues and organs ex vivo, producing organ and tissue scaffolds or frameworks composed of extracellular matrix containing intact vasculature suitable for recellularization. The present disclosure comprehends materials and methods for the decellularization of tissues or organs, materials and methods for maintenance of the tissue or organ scaffold, and materials and methods for the recellularization of such tissue or organ scaffolds.

In one embodiment, the step of providing a tissue sample or organ sample disclosed herein comprises dissecting a heart. In another embodiment, the step of providing a tissue sample or organ sample disclosed herein comprises dissecting a mammalian heart. In another embodiment, the step of providing a tissue sample or organ sample disclosed herein comprises dissecting an ovine heart. In another embodiment, the step of providing a tissue sample or organ sample disclosed herein comprises dissecting a porcine heart. In another embodiment, the step of providing a tissue sample or organ sample disclosed herein comprises dissecting a human heart.

In one embodiment, the dissected heart disclosed herein comprises a right heart flap. In another embodiment, the dissected heart disclosed herein comprises a left heart flap. In another embodiment, the dissected heart disclosed herein comprises a portion of the heart. In another embodiment, the dissected heart disclosed herein comprises the entire heart.

In one embodiment, the dissected heart disclosed herein comprises the vascular pedicle. In another embodiment, the dissected heart disclosed herein comprises the adjoining vascular pedicle. In another embodiment, the dissected heart disclosed herein comprises the pedicle. In another embodiment, the dissected heart disclosed herein comprises the adjoining pedicle. In another embodiment, the dissected heart disclosed herein comprises the vasculature. In another embodiment, the dissected heart disclosed herein comprises the adjoining vasculature.

In one embodiment, the dissected heart disclosed herein comprises the right coronary artery. In another embodiment, the dissected heart disclosed herein comprises the adjoining right coronary artery. In another embodiment, the dissected heart disclosed herein comprises the coronary artery. In another embodiment, the dissected heart disclosed herein comprises the adjoining coronary artery. In another embodiment, the dissected heart disclosed herein comprises one or more arteries. In another embodiment, the dissected heart disclosed herein comprises one or more adjoining arteries.

In one embodiment, the step of providing a tissue sample or organ sample disclosed herein is performed under sterile conditions.

In one embodiment, the step of providing a tissue sample or organ sample disclosed herein is performed by whole-organ irrigation.

In one embodiment, the whole-organ irrigation disclosed herein comprises using about 50 liters of heparinized cold normal saline. In another embodiment, the whole-organ irrigation disclosed herein comprises using about 50 liters of saline. In one embodiment, the whole-organ irrigation disclosed herein comprises using about 1-50 liters of heparinized cold normal saline. In another embodiment, the whole-organ irrigation disclosed herein comprises using about 1-50 liters of saline. In another embodiment, the whole-organ irrigation disclosed herein comprises using between 1 liter and 200 liters of heparinized cold normal saline. In another embodiment, the whole-organ irrigation disclosed herein comprises using between 1 liter and 200 liters of saline.

In one embodiment, the whole-organ irrigation disclosed herein comprises using about 50 liters of heparinized cold normal saline, following a single dose of heparin at about 400 IU/kg. In another embodiment, the whole-organ irrigation disclosed herein comprises using about 50 liters of saline, following a single dose of heparin at about 400 IU/kg. In one embodiment, the whole-organ irrigation disclosed herein comprises using about 1-50 liters of heparinized cold normal saline, following a single dose of heparin at about 400 IU/kg. In another embodiment, the whole-organ irrigation disclosed herein comprises using about 1-50 liters of saline, following a single dose of heparin at about 400 IU/kg. In another embodiment, the whole-organ irrigation disclosed herein comprises using between 1 liter and 200 liters of heparinized cold normal saline, following a single dose of heparin at about 400 IU/kg. In another embodiment, the whole-organ irrigation disclosed herein comprises using between 1 liter and 200 liters of saline, following a single dose of heparin at about 400 IU/kg.

In one embodiment, the whole-organ irrigation disclosed herein comprises using a single dose of heparin at between 10 IU/kg to 800 IU/kg. In another embodiment, the whole-organ irrigation disclosed herein comprises using a single dose of heparin at about 400 IU/kg.

In one embodiment, following the step of dissecting the tissue sample or organ sample disclosed herein the tissue sample or organ sample is cannulated. In another embodiment, following the step of dissecting the mammalian tissue sample or mammalian organ sample disclosed herein the mammalian tissue sample or mammalian organ sample is cannulated. In another embodiment, following the step of dissecting the cardiac tissue sample or heart sample disclosed herein the cardiac tissue sample or heart sample is cannulated. In another embodiment, following the step of dissecting the mammalian cardiac tissue sample or mammalian heart sample disclosed herein the mammalian cardiac tissue sample or mammalian heart sample is cannulated.

In one embodiment, following the step of cannulating the step of perfusing the tissue sample or organ sample is performed. In another embodiment, following the step of cannulating the step of perfusing the mammalian tissue sample or mammalian organ sample is performed. In another embodiment, following the step of cannulating the step of perfusing the mammalian cardiac tissue sample or mammalian heart sample is performed.

In one embodiment, the step of perfusing the tissue sample or organ sample disclosed herein is performed with distilled water. In another embodiment, the step of perfusing the mammalian tissue sample or mammalian organ sample disclosed herein is performed with distilled water. In another embodiment, the step of perfusing the mammalian cardiac tissue sample or mammalian heart sample disclosed herein is performed with distilled water.

In one embodiment, the step of perfusing the tissue sample or organ sample disclosed herein is performed with a solution. In another embodiment, the step of perfusing the mammalian tissue sample or mammalian organ sample disclosed herein is performed with a solution. In another embodiment, the step of perfusing the mammalian cardiac tissue sample or mammalian heart sample disclosed herein is performed with a solution.

In one embodiment, the step of perfusing with distilled water disclosed herein is performed for 1-5 hours. In one embodiment, the step of perfusing with distilled water disclosed herein is performed for about 1-5 hours. In one embodiment, the step of perfusing with distilled water disclosed herein is performed for 5 hours. In one embodiment, the step of perfusing with distilled water disclosed herein is performed for about 5 hours. In another embodiment, the step of perfusing with distilled water disclosed herein is performed for between 1 and 10 hours. In another embodiment, the step of perfusing with distilled water disclosed herein is performed for between 0.5 and 15 hours.

In one embodiment, the step of perfusing the tissue sample or organ sample with distilled water disclosed herein removes blood and thromboemboli from the tissue sample or organ sample. In another embodiment, the step of perfusing the cardiac tissue sample or heart sample with distilled water disclosed herein removes blood and thromboemboli from the cardiac tissue sample or heart sample. In another embodiment, the step of perfusing the tissue sample or organ sample with distilled water disclosed is performed to remove blood and thromboemboli from the tissue sample or organ sample. In another embodiment, the step of perfusing the cardiac tissue sample or heart sample with distilled water disclosed herein is performed to remove blood and thromboemboli from the cardiac tissue sample or heart sample.

In one embodiment, the step of perfusing the tissue sample or organ sample with distilled water disclosed herein is followed by an additional step of perfusing with another solution. In another embodiment, the step of perfusing the cardiac tissue sample or heart sample with distilled water disclosed herein is followed by an additional step of perfusing with another solution.

In one embodiment, the second perfusing step disclosed herein is performed for 10-250 hours. In another embodiment, the second perfusing step disclosed herein is performed for about 200 hours. In another embodiment, the second perfusing step disclosed herein is performed for about 216 hours. In another embodiment, the second perfusing step disclosed herein is performed for between 100 to 400 hours. In another embodiment, the second perfusing step disclosed herein is performed for between 50 to 500 hours.

In one embodiment, the second perfusing step disclosed herein is performed with a flow rate of approximately 40 ml/min. In another embodiment, the second perfusing step disclosed herein is performed with a flow rate of about 40 ml/min. In another embodiment, the second perfusing step disclosed herein is performed with a flow rate of between 20 ml/min to 60 ml/min. In another embodiment, the second perfusing step disclosed herein is performed with a flow rate of between 20 ml/min to 120 ml/min.

In one embodiment, any of the perfusing steps disclosed herein are performed via the right coronary artery. In another embodiment, any of the perfusing steps disclosed herein are performed via the right coronary arteries of the heart flaps. In another embodiment, any of the perfusing steps disclosed herein are performed via the arteries of the heart flaps. In another embodiment, any of the perfusing steps disclosed herein are performed via the arteries. In another embodiment, any of the perfusing steps disclosed herein are performed via the right heart flap.

In one embodiment, following the second perfusing step disclosed herein the tissue sample or organ sample is rinsed. In another embodiment, following the second perfusing step disclosed herein the cardiac tissue sample or heart sample is rinsed.

In one embodiment, following the second perfusing step disclosed herein the tissue sample or organ sample is rinsed with a solution. In another embodiment, following the second perfusing step disclosed herein the cardiac tissue sample or heart sample is rinsed with a solution. In another embodiment, following the second perfusing step disclosed herein the tissue sample or organ sample is rinsed with a solution of phosphate buffered saline (PBS), containing 100 U/ml penicillin and 100 U/ml streptomycin. In another embodiment, following the second perfusing step disclosed herein the cardiac tissue sample or heart sample is rinsed with a solution of phosphate buffered saline (PBS), containing 100 U/ml penicillin and 100 U/ml streptomycin. In another embodiment, following the second perfusing step disclosed herein the tissue sample or organ sample is rinsed with a solution of phosphate buffered saline (PBS), containing about 100 U/ml penicillin and about 100 U/ml streptomycin. In another embodiment, following the second perfusing step disclosed herein the cardiac tissue sample or heart sample is rinsed with a solution of phosphate buffered saline (PBS), containing about 100 U/ml penicillin and about 100 U/ml streptomycin.

In one embodiment, the step of rinsing disclosed herein is performed for 20 hours to 240 hours. In another embodiment, the step of rinsing disclosed herein is performed for about 20 hours to about 240 hours. In one embodiment, the step of rinsing disclosed herein is performed for 240 hours or about 240 hours. In another embodiment, the step of rinsing disclosed herein is performed for any value between 100 hours and 300 hours.

In one embodiment, the step of rinsing disclosed herein removes any residual chemicals. In another embodiment, the step of rinsing disclosed herein is performed to any residual chemicals.

In one embodiment, any solutions disclosed herein comprise antimycotic solutions and 1% (v/v) antibiotic. In one embodiment, any solutions disclosed herein in any step comprise antimycotic solutions and 1% (v/v) antibiotic. In one embodiment, any solutions disclosed herein comprise antimycotic solutions and about 1% (v/v) antibiotic. In one embodiment, any solutions disclosed herein in any step comprise antimycotic solutions and about 1% (v/v) antibiotic. In one embodiment, any solutions disclosed herein comprise antimycotic solutions and between 0.1% to 4% (v/v) antibiotic. In one embodiment, any solutions disclosed herein in any step comprise antimycotic solutions and between 0.1% to 4% (v/v) antibiotic.

In one embodiment, the decellularized tissue matrix or the decellularized organ matrix is fixed in solution to preserve the ECM. In another embodiment, the decellularized tissue matrix or the decellularized organ matrix is fixed in solution of 10% neutral buffered formalin to preserve the ECM. In another embodiment, the decellularized tissue matrix or the decellularized organ matrix is fixed in solution of about 10% neutral buffered formalin to preserve the ECM. In another embodiment, the decellularized tissue matrix or the decellularized organ matrix is fixed in solution of between 1% and 20% neutral buffered formalin to preserve the ECM.

In one embodiment, the solution preserving the decellularized tissue matrix or the decellularized organ matrix is effective for 24 hours. In another embodiment, the solution preserving the decellularized tissue matrix or the decellularized organ matrix is effective for about 24 hours. In another embodiment, the solution preserving the decellularized tissue matrix or the decellularized organ matrix is effective for up to 2 weeks.

In one embodiment, preserving the decellularized tissue matrix or the decellularized organ matrix prevents microbial contamination of the matrix. In another embodiment, preserving the decellularized tissue matrix or the decellularized organ matrix by treatment with an antibiotic solution or cocktail prevents microbial contamination of the matrix.

In one embodiment, the decellularized tissue matrix or the decellularized organ matrix described herein are recellularized with cardiac cells, mesentrial stem cells or other defriciated cells that would provide appropriate recellularization. In another embodiment, the decellularized tissue matrix or the decellularized organ matrix described herein are recellularized with cells. In another embodiment, the decellularized tissue matrix or the decellularized organ matrix described herein are recellularized with mammalian cardiac cells. In another embodiment, the decellularized tissue matrix or the decellularized organ matrix described herein are recellularized with mammalian cells. In another embodiment, the decellularized tissue matrix or the decellularized organ matrix described herein are recellularized with rodent cardiac cells. In another embodiment, the decellularized tissue matrix or the decellularized organ matrix described herein are recellularized with rodent cells. In another embodiment, the decellularized tissue matrix or the decellularized organ matrix described herein are recellularized with cardiac cells mesentrial stem cells or other defriciated cells that would provide appropriate recellularization. In another embodiment, the decellularized tissue matrix or the decellularized organ matrix described herein are recellularized with rat cells.

In one embodiment, the decellularized tissue matrix or the decellularized organ matrix described herein are recellularized with cardiac cells form neonatal green fluorescent protein (GFP) positive newborn rats. In another embodiment, the decellularized tissue matrix or the decellularized organ matrix described herein are recellularized with cells form GFP positive newborn rats. In another embodiment, the decellularized tissue matrix or the decellularized organ matrix described herein are recellularized with cardiac cells form GFP positive rats. In another embodiment, the decellularized tissue matrix or the decellularized organ matrix described herein are recellularized with cells form GFP positive rats. In another embodiment, the decellularized tissue matrix or the decellularized organ matrix described herein are recellularized with cardiac cells form GFP positive newborn rats at the age of postnatal day 1-3. In another embodiment, the decellularized tissue matrix or the decellularized organ matrix described herein are recellularized with cells form GFP positive newborn rats at the age of postnatal day 1-3.

In one embodiment, the decellularized tissue matrix or the decellularized organ matrix described herein are recellularized with GFP-positive cardiac cells.

In one embodiment, the decellularized tissue matrix or the decellularized organ matrix described herein are recellularized with cells isolated from hearts. In another embodiment, the decellularized tissue matrix or the decellularized organ matrix described herein are recellularized with cells isolated from mammalian hearts. In another embodiment, the decellularized tissue matrix or the decellularized organ matrix described herein are recellularized with cells isolated from rodent hearts. In another embodiment, the decellularized tissue matrix or the decellularized organ matrix described herein are recellularized with cells isolated from rat hearts.

In one embodiment, the cells isolated for recellularization are maintained in solution to preserve the cells. In another embodiment, the cells isolated for recellularization are maintained in Dulbecco's Modified Eagle Medium (DMEM) with 10% fetal bovine serum (FBS) at 37° C. in a CO2 incubator to preserve the cells. In another embodiment, the cells isolated for recellularization are maintained in DMEM with about 10% fetal bovine serum (FBS) at about 37° C. in a CO2 incubator to preserve the cells. In another embodiment, the cells isolated for recellularization are maintained in DMEM. In another embodiment, the cells isolated for recellularization are maintained in DMEM with 10% fetal bovine serum (FBS). In another embodiment, the cells isolated for recellularization are maintained at about 37° C. In another embodiment, the cells isolated for recellularization are maintained in a CO2 incubator. In another embodiment, the cells isolated for recellularization are maintained in a humidified atmosphere containing 5% CO2. In another embodiment, the cells isolated for recellularization are maintained in a humidified atmosphere containing about 5% CO2. In another embodiment, the cells isolated for recellularization are maintained in a humidified atmosphere containing between 1% and 10% CO2. In another embodiment, the cells isolated for recellularization are maintained in a humidified atmosphere containing between 5% and 10% CO2.

In one embodiment, the decellularized cardiac flaps disclosed herein undergo a gamma irradiation sterilization process before the cell seeding. In another embodiment, the decellularized cardiac flaps disclosed herein undergo a gamma radiation sterilization before the cell seeding. In one embodiment, the decellularized tissue matrix or decellularized organ matrix undergo gamma sterilization process before the cell seeding. In one embodiment, the decellularized cardiac tissue matrix or decellularized heart matrix undergo gamma sterilization process before the cell seeding.

In one embodiment, the first step in the in-vitro recellularization of the decellularized tissue matrix or decellularized matrix starts with connecting the matrices to a closed-system bioreactor that was placed in a cell culture incubator with 5% CO2 at 37° C. for the purpose of cardiac cells seeding. In another embodiment, the first step in the in-vitro recellularization of the decellularized tissue matrix or decellularized matrix starts with connecting the matrices to a closed-system bioreactor. In one embodiment, the first step in the in-vitro recellularization of the decellularized tissue matrix or decellularized matrix starts with connecting the matrices to a closed-system bioreactor that was placed in a cell culture incubator. In one embodiment, the first step in the in-vitro recellularization of the decellularized tissue matrix or decellularized matrix starts with connecting the matrices to a closed-system bioreactor that was placed in a cell culture incubator with about 5% CO2. In one embodiment, the first step in the in-vitro recellularization of the decellularized tissue matrix or decellularized matrix starts with connecting the matrices to a closed-system bioreactor that was placed in a cell culture incubator at about 37° C. In one embodiment, the first step in the in-vitro recellularization of the decellularized tissue matrix or decellularized matrix starts with connecting the matrices to a closed-system bioreactor that was placed in a cell culture incubator with between 1% and 10% CO2. In one embodiment, the first step in the in-vitro recellularization of the decellularized tissue matrix or decellularized matrix starts with connecting the matrices to a closed-system bioreactor that was placed in a cell culture incubator at between 30° C. and 47° C.

In one embodiment, the decellularized heart flaps are preserved in a container and the medium is circulated with 10 mL/min via the right coronary of the flaps for 24 h. In another embodiment, the decellularized heart flaps are preserved in a container and the medium is circulated with 10 mL/min. In one embodiment, the decellularized heart flaps are preserved in a container and the medium is circulated via the right coronary of the flaps. In one embodiment, the decellularized heart flaps are preserved in a container for 24 h. In one embodiment, the decellularized heart flaps are preserved in a container and the medium is circulated with about 10 mL/min via the right coronary of the flaps for about 24 h. In another embodiment, the decellularized heart flaps are preserved in a container and the medium is circulated with about 10 mL/min. In one embodiment, the decellularized heart flaps are preserved in a container for about 24 h. In one embodiment, the decellularized heart flaps are preserved in a container and the medium is circulated with about between 1 mL/min to 40 mL/min via the right coronary of the flaps for between 1 hour and 48 hours. In another embodiment, the decellularized heart flaps are preserved in a container and the medium is circulated with between 1 mL/min to 40 mL/min. In one embodiment, the decellularized heart flaps are preserved in a container for between 1 hour and 48 hours.

In one embodiment, the decellularized heart flaps are removed from the growth medium prior to perfusion.

In one embodiment, the decellularized heart flaps are perfused with ten million GFP⁺ rat neonatal cardiac cells via the right coronary of the cardiac flaps. In another embodiment, the decellularized heart flaps are perfused with ten million GFP⁺ rat neonatal cardiac cells. In one embodiment, the decellularized heart flaps are perfused with about ten million GFP⁺ rat neonatal cardiac cells. In another embodiment, the decellularized heart flaps are perfused with between 1 and 20 million GFP⁺ rat neonatal cardiac cells. In another embodiment, the decellularized heart flaps are perfused via the right coronary of the cardiac flap.

In one embodiment, the decellularized heart flaps are perfused with ten million cardiac cells via the right coronary of the cardiac flaps. In another embodiment, the decellularized heart flaps are perfused with ten million cardiac cells. In one embodiment, the decellularized heart flaps are perfused with about ten million cardiac cells. In another embodiment, the decellularized heart flaps are perfused with between 1 and 20 million cardiac cells.

In one embodiment, following perfusion with cardiac cells the heart flaps are injected with 5 million GFP+ rat neonatal cardiac cells into five different areas of the myocardium on day 5 and day 10. In another embodiment, following perfusion with cardiac cells the heart flaps are injected with 5 million GFP+ rat neonatal cardiac cells at two time points. In another embodiment, following perfusion with cardiac cells the heart flaps are injected with about 5 million GFP+ rat neonatal cardiac cells. In another embodiment, following perfusion with cardiac cells the heart flaps are injected with between 1 and 15 million GFP+ rat neonatal cardiac cells.

In one embodiment, the total amount of cells injected into the decellularized cardiac flap is dependent upon the size of the flap.

In one embodiment, following perfusion with cardiac cells the heart flaps are injected with GFP+ rat neonatal cardiac cells into five different areas of the myocardium. In another embodiment, following perfusion with cardiac cells the heart flaps are injected with GFP+ rat neonatal cardiac cells into about five different areas of the myocardium. In another embodiment, following perfusion with cardiac cells the heart flaps are injected with GFP+ rat neonatal cardiac cells into between 1 and 15 different areas of the myocardium.

In one embodiment, following perfusion with cardiac cells the heart flaps are injected with GFP+ rat neonatal cardiac cells into five different areas of the heart flaps. In another embodiment, following perfusion with cardiac cells the heart flaps are injected with GFP+ rat neonatal cardiac cells into about five different areas of the heart flaps. In another embodiment, following perfusion with cardiac cells the heart flaps are injected with GFP+ rat neonatal cardiac cells into between 1 and 15 different areas of the heart flaps.

In one embodiment, following perfusion with cardiac cells the heart flaps are injected with cardiac cells day 5 and day 10. In another embodiment, following perfusion with cardiac cells the heart flaps are injected with cardiac cells at two time points. In another embodiment, following perfusion with cardiac cells the heart flaps are injected with cardiac cells at two time points selected between day 5 and day 20. In another embodiment, following perfusion with cardiac cells the heart flaps are injected with cardiac cells at one time point. In another embodiment, following perfusion with cardiac cells the heart flaps are injected with cardiac cells at any time points selected between day 5 and day 20.

In one embodiment, disclosed herein is a method of producing a recellularized cardiac flap by repopulating a decellularized cardiac flaps with GFP positive rat neonatal cardiac cells, the method comprising: (a) connecting the decellularized cardiac flaps to a closed-system bioreactor that is placed in a cell culture incubator with about 5% CO2 at about 37° C., (b) circulating the medium with 10 mL/min via the right coronary of said cardiac flap for about 24 h, (c) removing the decellularized cardiac flaps, (d) perfusing the flaps with ten million GFP+ rat neonatal cardiac cells via the right corner of the cardiac flaps, (e) injecting 5 million GFP+ rat neonatal cardiac cells into five different areas of the myocardium on day 5 and day 10, thereby producing the recellularized cardiac flap.

In one embodiment, the growth medium perfusions disclosed herein are started after a 4 hour interval. In another embodiment, the growth medium perfusions disclosed herein are started after about a 4 hour interval. In another embodiment, the growth medium perfusions disclosed herein are started after between a 1 hour and 12 hour interval.

In one embodiment, the growth medium perfusion disclosed herein attaches said cells to the decellularized cardiac flaps.

In one embodiment, the recellularized methods disclosed herein are performed on a shaker. In another embodiment, the recellularized methods disclosed herein are performed on a shaker which prevents cell adhesion to the bottle wall and decreases cardiac cell concentration. In another embodiment, the recellularized methods disclosed herein are performed on a shaker to prevent cell adhesion to the bottle wall and decrease cardiac cell concentration.

In one embodiment, the cell injection during the recellularized methods described herein are performed with a needle. In another embodiment, the injections described herein are performed with a needle.

In one embodiment, the medium used during the recellularization methods described herein is changed every 3 days. In another embodiment, the medium used during the recellularization methods described herein is changed about every 3 days. In another embodiment, the medium used during the recellularization methods described herein is changed at a time comprising between 1 and 10 days. In another embodiment, the medium used during the recellularization methods described herein is changed repeatedly at a time comprising between 1 and 10 days.

In one embodiment, the recellularized methods disclosed herein are performed for a total of 20 days. In another embodiment, the recellularized methods disclosed herein are performed for a total of about 20 days. In another embodiment, the recellularized methods disclosed herein are performed for a total of between 1 and 20 days. In another embodiment, the recellularized methods disclosed herein are performed for a total of between 1 and 40 days.

In one embodiment, disclosed herein are recellularized tissue, recellularized organs, or recellularized cardiac flaps produced by the recellularization methods disclosed herein.

In one embodiment, the recellularized tissue, recellularized organ, or recellularized cardiac flap disclosed herein are transplantable.

In one embodiment, any of decellularization processes described herein do not reduce the tensile strength of the decellularized right atrium. In one embodiment, any of decellularization processes described herein do not significantly reduce the tensile strength of the decellularized right atrium.

In one embodiment, there is no destructive changes in the construction of the vascular network in the decellularized flaps. In one embodiment, there is no significant destructive changes in the construction of the vascular network in the decellularized flaps.

In one embodiment, disclosed herein is a method of treating a heart aneurysm in a subject by grafting a recellularized cardiac patch or flap onto the heart, by replacing a damaged part of the heart with a the recellularized cardiac patch or flap, or by implanting a recellularized cardiac patch or flap on a ventricular surface.

In one embodiment, disclosed herein is a method of treating a myocardial infarction in a subject by grafting a recellularized cardiac patch or flap onto the heart, by replacing a damaged part of the heart with the recellularized cardiac patch or flap, or by implanting a recellularized cardiac patch or flap in ventricular aneurysm surface.

In one embodiment, disclosed herein is a method of preventing a heart aneurysm in a subject following a myocardial infarction by grafting a recellularized cardiac patch or flap onto the heart, by replacing a damaged part of the heart with the recellularized cardiac patch or flap, or by implanting a recellularized cardiac patch or flap on a ventricular surface.

In one embodiment, disclosed herein is a method of treating a heart aneurysm in a subject following a myocardial infarction by grafting a recellularized cardiac patch or flap onto the heart, by replacing a damaged part of the heart with the recellularized cardiac patch or flap, or by implanting a recellularized cardiac patch or flap on a ventricular aneurysm surface. In one embodiment, the grafting disclosed herein is coronary artery bypass graft. In another embodiment, the grafting disclosed herein is coronary bypass graft. In another embodiment, the grafting disclosed herein is an artery bypass graft.

In one embodiment, the subjects disclosed herein are human. In one embodiment, the subjects disclosed herein are male. In another embodiment, the subjects disclosed herein have a history of at least one myocardial infarction. In another embodiment, the subjects disclosed herein have a history of at least one myocardial infarction resulting in necrotic tissue on the heart. In another embodiment, the subjects disclosed herein have necrotic tissue on the heart. In another embodiment, the subjects disclosed herein have tissue damage on the heart. In another embodiment, the subjects disclosed herein have a damaged part of the heart that is an infarcted part of the heart.

In one embodiment, the heart aneurysm disclosed herein is a rupture.

In one embodiment, the grafted, replaced, or implanted recellularized cardiac patch promotes anastomosis. In another embodiment, the grafted, replaced, or implanted recellularized cardiac patch promotes anastomosis which then connects the recellularized cardiac patch to the aorta or other arteries.

In one embodiment, the in vitro seeded cells in the cardiac patch or flap described herein receive nutrients, oxygen, or new circulating bone marrow stem cells.

In another embodiment, the cardiac patch or flap described herein repairs the infarcted heart by neo-vascularization and supportive cardiac pump with very young non atherosclerotic arteries. In another embodiment, the cardiac patch or flap described herein repairs the infarcted heart by neo-vascularization. In another embodiment, the cardiac patch or flap described herein repairs the infarcted heart by neo-vascularization and supportive cardiac pump with non-atherosclerotic arteries.

In one embodiment, the cardiac patch or flap described herein prevents left ventricular ballooning and increases the cardiac output of the infarcted heart. In another embodiment, the cardiac patch or flap described herein prevents right ventricular ballooning and increases the cardiac output of the infarcted heart.

In one embodiment, the cardiac patch or flap described herein improves the distribution and effectiveness of prescribed cardiac medicines.

In one embodiment, the cardiac patch or flap described herein covers the infarcted aneurismal left or right ventricle and prevents cardiac aneurism rupture and/or sudden death

In one embodiment, the viable cardiac tissue in the cardiac patch or flap described herein harmonizes with the native host heart.

In one embodiment, the borders of the cardiac patch or flap described herein receive peripheral revascularization and angioneogenesis.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Having described preferred embodiments of the invention with reference to the accompanying drawings, it is to be understood that the invention is not limited to the precise embodiments, and that various changes and modifications may be affected therein by those skilled in the art without departing from the scope or spirit of the invention as defined in the appended claims.

### Example 1

### Coronary-Based Right Heart Flap Recellularization by Rat Neonatal Whole Cardiac Cells

Objective: To develop decellularized coronary-based right heart flaps by perfusion-based method followed by recellularization with rats' neonatal green fluorescent protein (GFP)-positive cardiac cells.

### Materials and Methods

### Preparation of Sheep Hearts

Fresh right hearts with attached pedicle and right coronary arteries were precisely dissected under sterile conditions from eight healthy and physiologically normal adult sheep (same age and gender) with a mean body weight of 35±6 kg. Whole-organ irrigation was applied using 50 liters of heparinized cold normal saline, following a single dose of heparin (400 IU/kg). NIH guidelines (or for non-U.S. residents similar national regulations) for the care and use of laboratory animals (NIH Publication #85-23 Rev. 1985) were observed. The care of animals was also in accordance with the guidelines for the care and use of laboratory animals (Medical Ethics and History of Medicine Research Center, Iran). This study was approved by the committee on animal welfare in the Tehran University of Medical Sciences.

### Perfusion-Based Decellularization Method

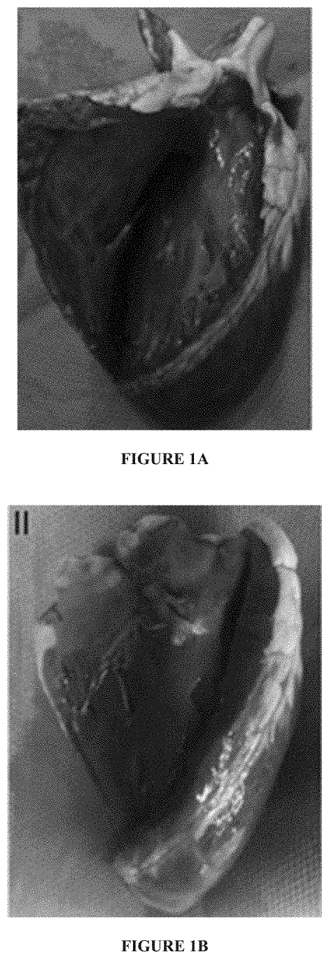

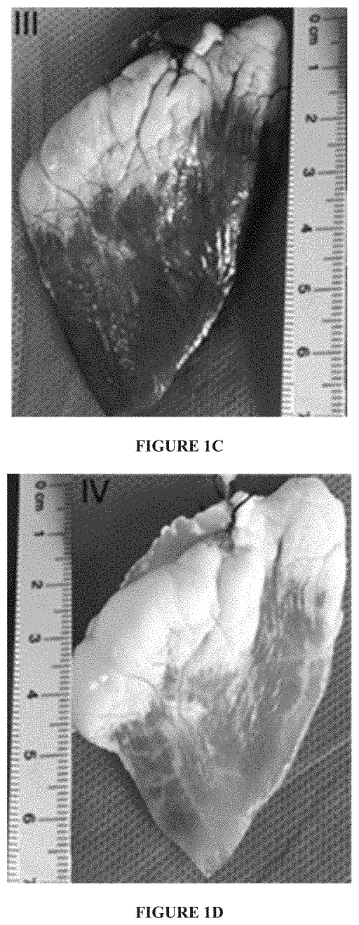

Immediately after heart removal, the right coronary arteries were cannulated. The right hearts were then perfused with distilled water for 5 h to remove the blood and thromboemboli, followed by perfusion of 1% SDS for 216 hours via right coronary arterycoroners of flaps with a flow rate of approximately 40 ml/min. All the samples were rinsed with phosphate buffered saline (PBS), containing 100 U/ml penicillin, 100 U/ml streptomycin (v/v; Sigma-Aldrich, MO, USA), for another 240 hours to remove any residual chemicals. Antimycotic solutions and 1% (v/v) antibiotic were used in all the previously mentioned solutions and under sterile conditions. FIG. 1D shows the right heart after following these decellularization methods.

### Perfusion-Decellularized Sheep Heart Matrix Characterization

The decellularized samples of both groups were fixed in 10% neutral buffered formalin (Merck, Darmstadt, Germany) for 24 hours to evaluate the competence of the decellularization protocol in removing cell nuclei and preservation of ECM. Tissue samples were paraffin embedded, sectioned into 5-8 μm thickness, and stained with hematoxylin-eosin (H&E) and 4, 6-diamidino-2-phenylindole (DAPI, Sigma-Aldrich). The amount of soluble collagen and sGAGs were measured in native and decellularized right ventricles (n=4) using Sircol® Soluble Collagen and Blyscan™ Glycosaminoglycan Assay Kits respectively (Biocolor, UK). Natural and decellularized cardiac flaps samples were also fixed with 2.5% glutaraldehyde and washed in PBS twice in order to assess the morphological features and structural integrity of the scaffolds by scanning electron microscopy (SEM). After putting the scaffolds in serial concentration of ethanol, they were processed under a critical point dryer (Autosamdri814; Tousimis) for 15 min. Gold sputter coating (Hitachi-54160, Japan) was used to enhance the conductivity of electrons and visualize the surfaces. The staining was performed according to the previously described method (Lee J-B, Mintz G S, Lisauskas J B, Biro S G, Pu J, Sum S T et al. Histopathologic validation of the intravascular ultrasound diagnosis of calcified coronary artery nodules. Am J Cardiol. 2011; 108(11):1547-51). A Nikon digital camera DXM 1200 (Amsterdam, Netherlands) was used for taking the images. Image analysis was performed by using the application of Photoshop 10.0 software (Adobe Systems, Inc., Mountain View, Calif., USA) and Image Pro (Image Pro Inc., Boston, Mass., USA). According to manufacturer's instructions, a Genomic DNA Purification Kit (Thermo Fisher Scientific, MA, USA) was used for total genomic DNA extraction from native and decellularized tissues (30-40 mg). DNA contents quantified using a NanoDrop ND-1000 spectrophotometer (NanoDrop Technologies, DE, USA) and expressed as ng/mg of dry tissue (Scarritt M E, Pashos N C, Bunnell B A, A review of cellularization strategies for tissue engineering of whole organs. Front Bioeng Biotechnol. 2015; 3:43). The tensile properties of decellularized and native atrium and ventricle were also examined. Universal test machine (Zwick/roll, Germany model Hct 400/25 with 25 KN load cell, software toolkit 1998) was used for the procedure. The specimens were cut into strips in the size of 20-mm long×15-mm wide×0.46-mm thick sections. A constant elongation rate of 0.5 mm/s was applied for clench the specimens with sample holders. The system drew the curve for strength-stress and the maximal point indicated the utmost pressure tolerance. For computed tomography angiography (CTA) imaging, isosmolar contrast medium Visipaque™ (iodixanol) was injected into decellularized scaffolds via cannulated coronary arteries with a flow rate of 3 ml/min. A GE CT 16 Slice Bright Speed CT scanner machine (GE Healthcare, Little Chalfont, UK) was used for analysis of the images.

### Cell Culture

Heart tissues were aseptically acquired from fifteen GFP positive newborn rats at the age of postnatal day 1-3, and rinsed with fresh, sterile Hanks' Balanced Salt Solution (HBSS). After dissection of unwanted parts, the remained tissues were transferred to fresh ice-cold filtered HBSS buffer+0.05% trypsin and minced. When the pieces are the size of around 0.5 mm³ to about 1 mm³, they are then ready. The dish was closed and wrapped entirely in parafilm. The wrapped petri dish was placed in cold room (4° C.) and left on a shaker overnight. In the next step, heart pieces were collected and located in a 50 mL falcon tube with 10 mL medium (DMEM with 10% FBS). The falcon was swirled slowly for 7 min (roughly 60 rpm) in water bath 37° C. to inactive trypsin. Then collagenase type II, (0.1%) for 3 times was added to tissues. After that, harvested cells were performed by centrifugation for 5 min at 1000 rpm. Cell pellet was then resuspended in 2 mL warm culture medium. Cells isolated from hearts were seeded in 175 culture flask. Cells were incubated in incubator for 1 h for attachment of non-cardiomyocytes and remaining the cardiomyocytes in culture medium. The suspended cells were plated into a new tissue culture flask. The medium of non-cardiomyocytes was changed after 3 hours and then the next day early in the morning and for the cardiomyocytes was changed the next day. After that the culture mediums were replaced with fresh media for every 48 h to reach 25×10⁶ cells.

MTT Viability Assay

MTT assay was done for evaluating the effect of decellularized ECM cytotoxicity on cell viability (Kumar P, Nagarajan A, Uchil P D. Analysis of cell viability by the MTT assay. Cold Spring Harb Protoc. 2018; 2018(6):pdb. prot095505). For this purpose, GFP⁺ rat cardiac cells were seeding into wells of flat-bottomed %-well plates at 1×10⁴ cells/well (in DMEM with 10% FBS) and incubated for 24 h at 37° C. Then the decellularized heart slices (2 mm²) were added to the culture plate and incubated for 48 h at 37° C. After that 10 μL of the 12 mM MTT stock solution added to each well and incubated at 37° C. for 4 hours then, 50 μL of Dimethyl sulfoxide (DMSO) was added to each well and mixed. The optical density (OD) absorbance at 570 nm was determined with use of a microplate reader (RT-6000, Rayto, USA). A comparison between the viability of grown cells in scaffold-conditioned and control medium was defined as cell viability. Ten replicates were considered per sample.

### In-Vitro Recellularization of Perfusion-Based Decellularized Flaps

The decellularized cardiac flaps underwent gamma radiation sterilization process before the cell seeding. Decellularized cardiac flaps were connected to a closed-system bioreactor that was placed in a cell culture incubator with 5% CO2 at 37° C. for the purpose of cardiac cells seeding. The decellularized hearts flaps were preserved in the container and the medium (DMEM with 10% FBS) was circulated with 10 mL/min via the right coronary artery of the flaps for 24 h at. Then the scaffolds removed from the bioreactor and ten million GFP⁺ rat neonatal cardiac cells were perfused via the right coronary artery and the growth medium (DMEM with 10% FBS) perfusion was started after a 4 h interval to let the cells attach to the decellularized cardiac flaps. For two times, day 5 and day 10, and 5 million GFP rat neonatal cardiac cells were needle-injected into five different areas of the myocardium each time. The medium was changed every 3 days and perfusion of growth medium was restarted 4 hours after each cell injection. All the steps of the recellularization method were performed on a shaker, in order to prevent cell adhesion to the bottle wall and decrease of cardiac cell concentration. Subsequently, recellularized flaps were precisely removed from the bioreactor after 20 days for further histological examinations. For immunohistochemical examinations, paraffin embedded sections were heated 2.5 min at 850 watt and 10 min at 160 watt in antigen retrieval Citra solution (Biogenex, Klinipath, Belgium). Slides were flooded in 30% (v/v) rabbit serum in PBS for 30 min at room temperature. Then, they were incubated with primary antibodies, rinsed in a PBS solution containing 2% (v/v) bovine serum albumin for 1 h at 37 C. In immunohistochemical staining, CD34, α-SMA, desmin. and vimentin Antigen Antibodies (Santa Cruz Biotechnology) were applied. H&E staining was also used in recellularized flaps.

### Statistical Analysis

SPSS software (v.17, SPSS, Chicago, Ill., USA) was used for statistical analysis. All data were expressed as mean±standard deviation (SD). To compare two groups, T test was performed. A P value of <0.05 was considered statistically significant.

### Results Summary and Explanation for Examples

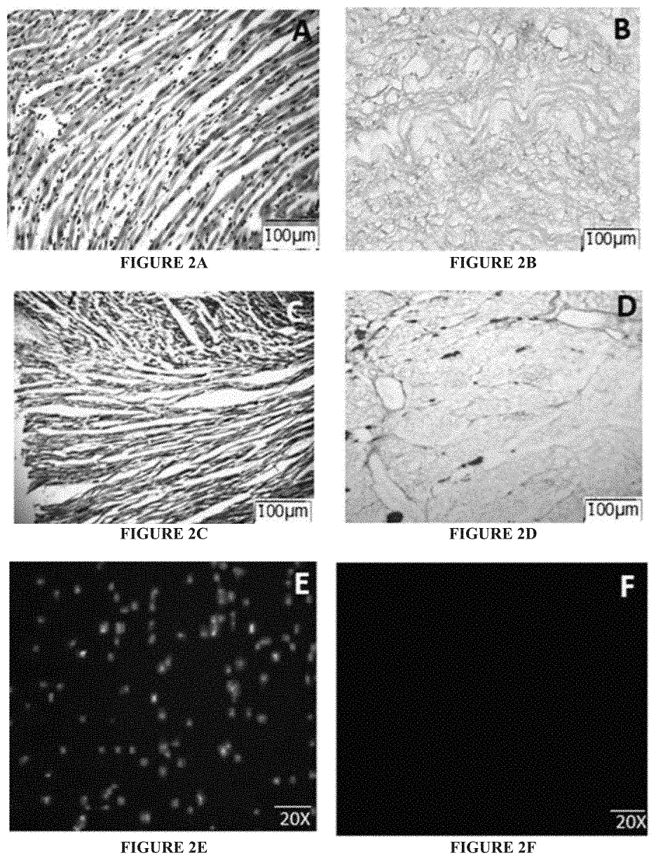

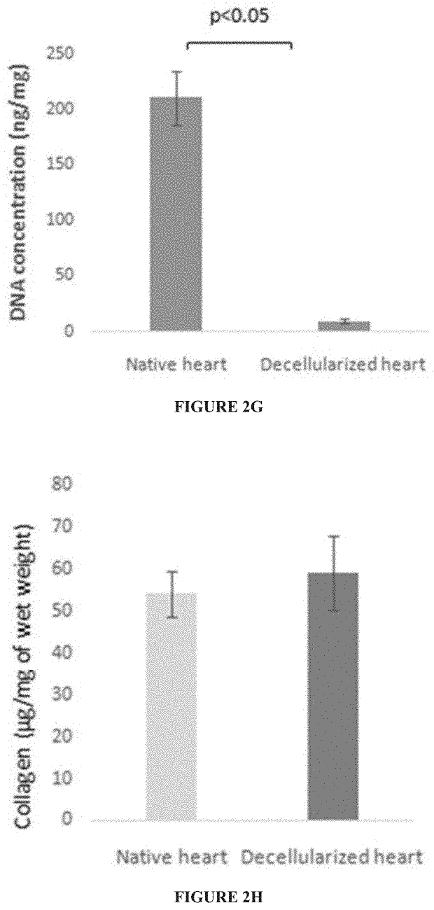

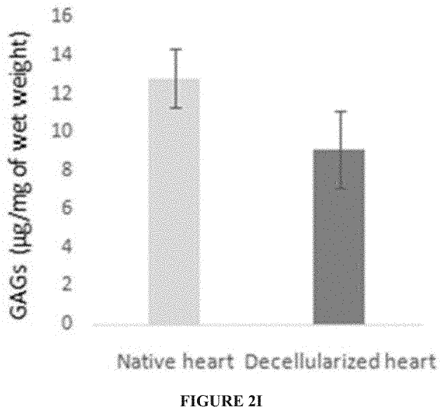

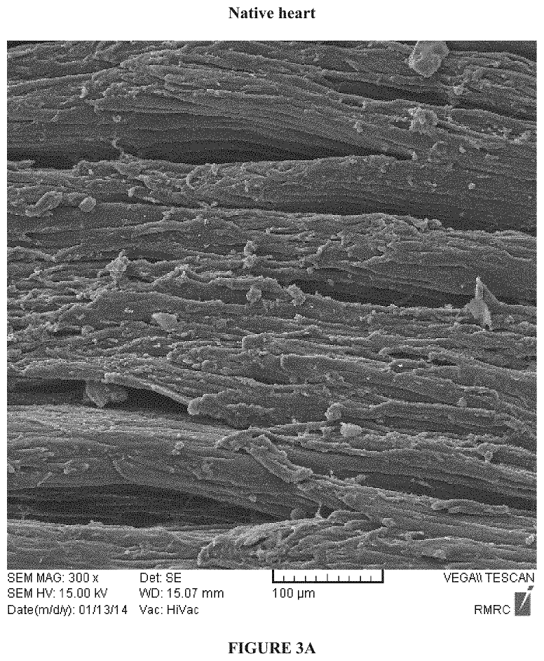

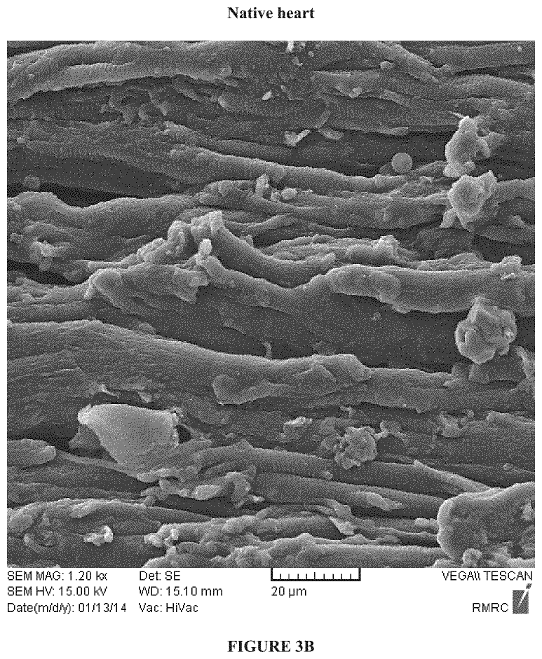

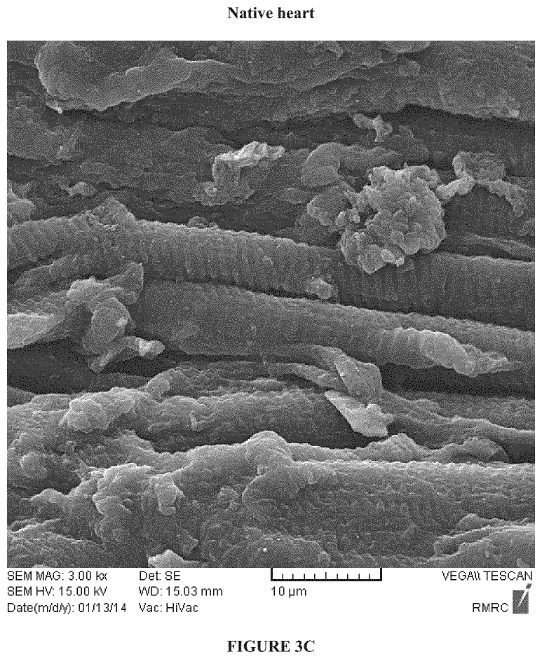

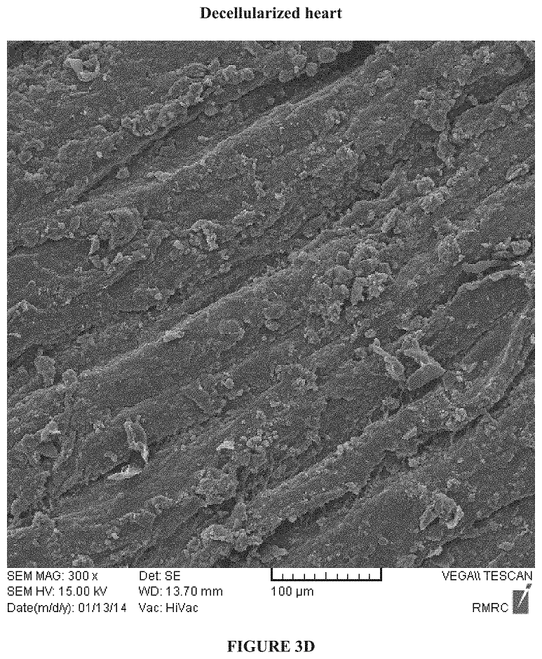

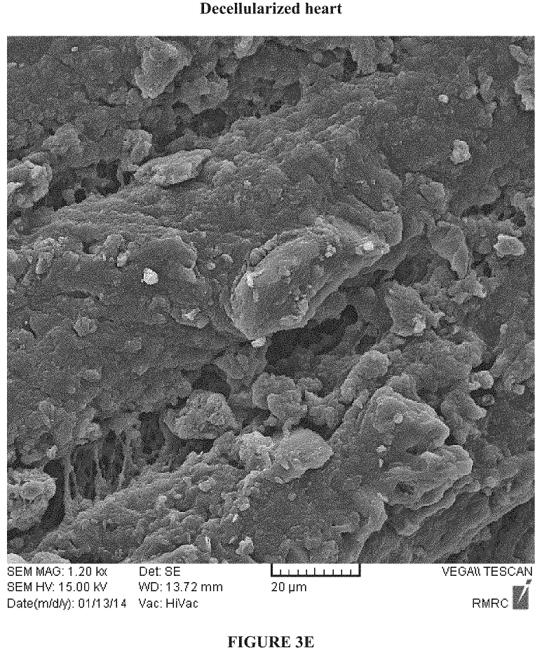

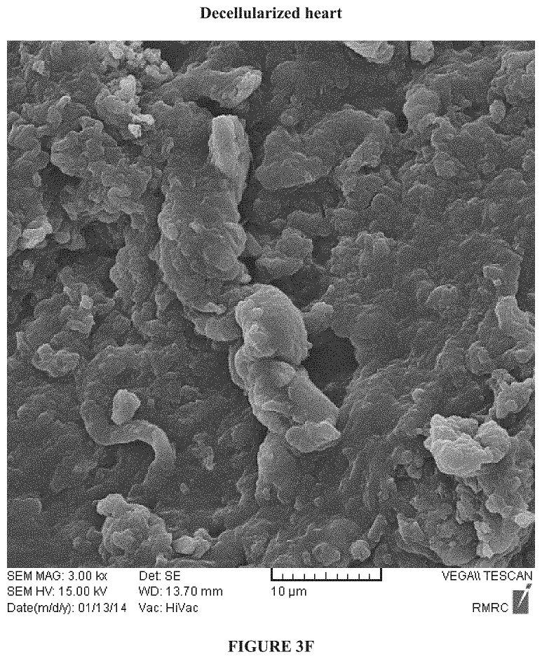

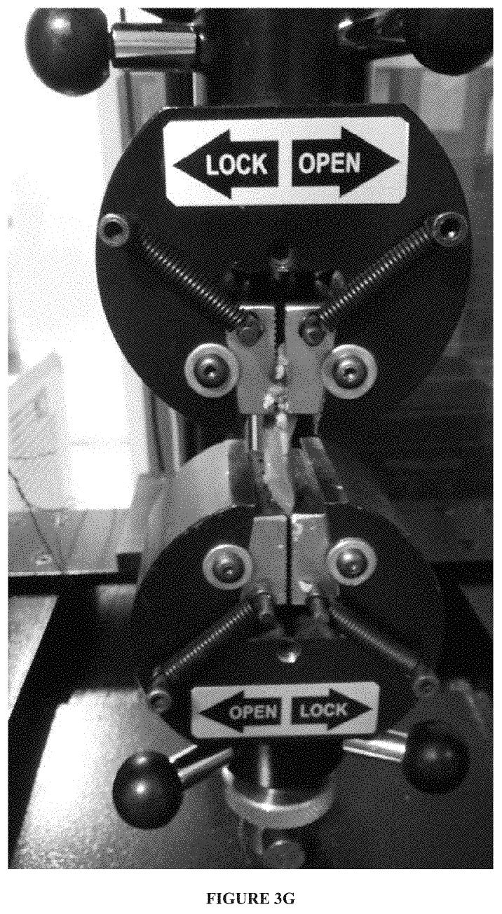

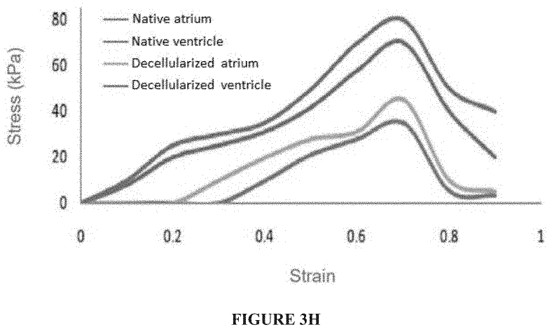

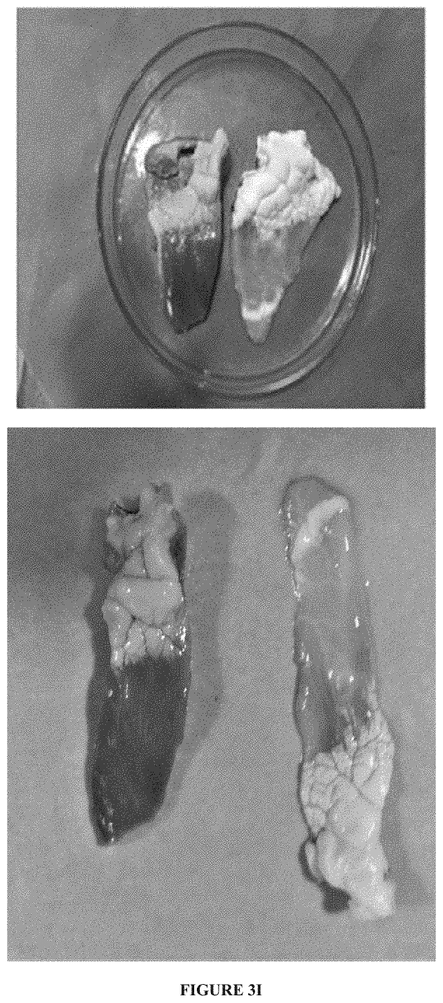

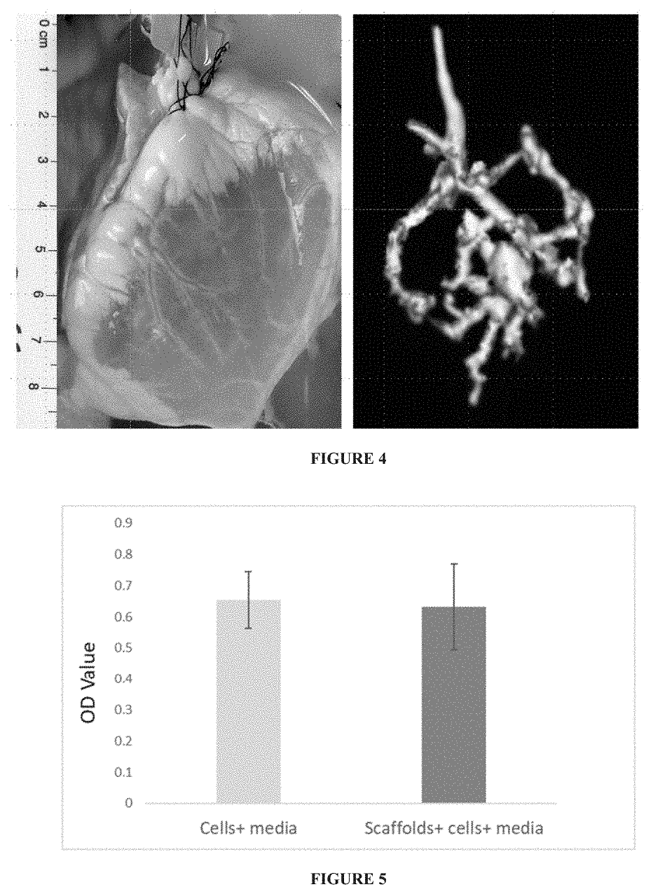

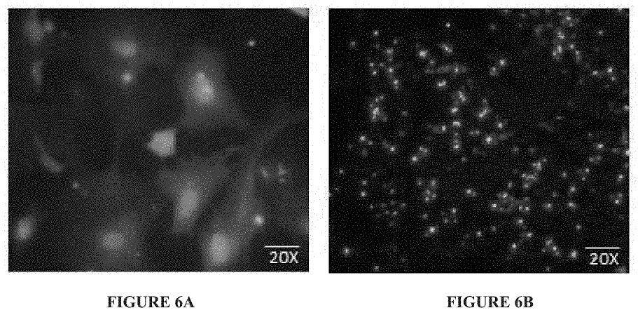

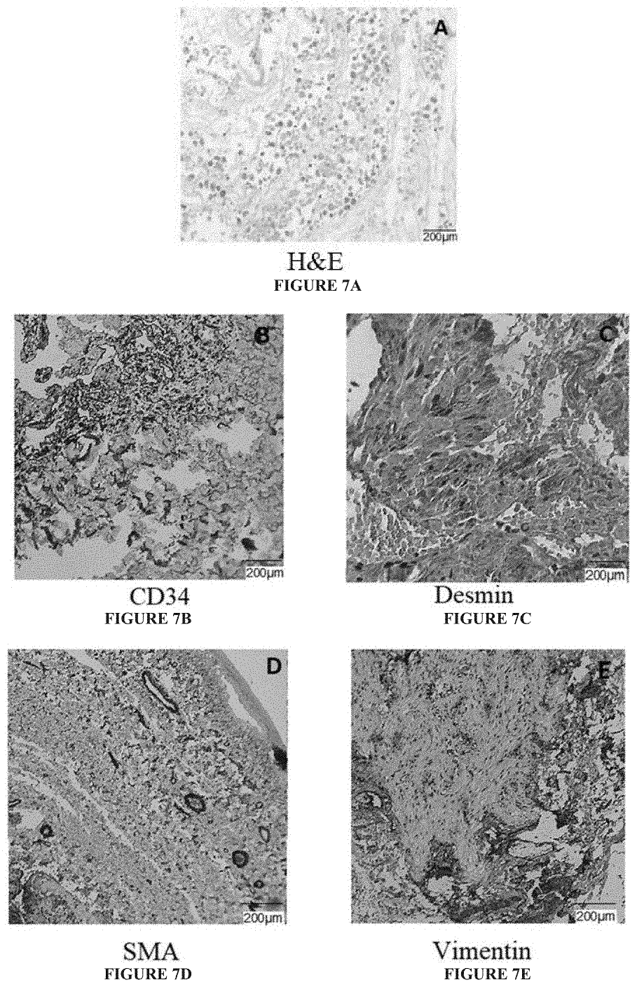

Decellularization process of the coronary-based right heart flap is depicted in FIGS. 1A-1D. H&E and trichrome staining also revealed a decellularized scaffold with well preserved ECM and well organized collagen fibers (FIG. 2A-2D). DAPI staining confirmed the effectiveness of the decellularization process with complete cell removal in decellularized right atrium and ventricle (as the thinnest part of the heart) as compared to the native tissues (FIG. 2E-2F). Additionally, there was an approximate 95% reduction (p<0.05) in the DNA contents of the decellularized scaffold compared with that of the native ones (9.43±2.35 vs 210±24.15 ng/mg of dry tissue, respectively) (FIG. 2G). Comparing the average amounts of soluble collagen and sGAGs between the native and decellularized right ventricle shows the non-significant (p>0.05) increase in collagen content of decellularized samples (59.07±8.85) when compare with the native one (54.24±5.34) (FIG. 2H) in contrast, there is a reduction in GAGs content after decellularization (9.11±2.05) when compare with the GAGs content of native specimens (12.82±1.52), the reduction is not significant (p>0.05)(FIG. 2I). The results of SEM evaluation depicted that the micro and ultrastructural characteristics of the scaffolds were maintained. The ECM network of all the decellularized segments was similar to that of the native heart (FIGS. 3A-3F). The results of the tensile test showed that there was no significant reduction in the decellularized right atrium (41±0.31 kPa) compared with the native control (34±0.62 kPa) (p>0.05). Same results were obtained for the right ventricle compared with the native one (81±0.35 kPa vs 72±0.61 kPa) (FIGS. 3G-3I). The preservation of the right coronary artery and vascular pedicle of the right heart after the decellularization process was pointed out in the analysis of CTA images (FIG. 4). Remarkably, the findings obtained from CTA demonstrated that there were no significant destructive changes in the construction of the vascular network in the decellularized flaps. The outcomes of the MTT assay showed that the existence of decellularized matrix in the culture medium did not have any cytotoxicity effect on the viability of the GFP⁺ rat cardiac cell. The cells grown in wells in the presence of decellularized flaps (0.65±0.09) showed no significant differences (p>0.05) in optic density (OD) values when compare with wells lacking decellularized flap (0.63±0.13) (FIG. 5). In-vitro cell seeding evaluation showed promising results in cell seeding process. The results of IF microscopy after recellularization process authenticated that GFP-positive cardiac cells were successfully seeded on the empty flaps after 20 days in a bioreactor (FIGS. 6A-6B). H&E staining of the recellularized flaps depicted that the structure was noticeably similar to the natural heart with well-organized architecture. Moreover, IHC staining showed that cardiomyocytes were seeded and repopulated the surrounding area of the scaffolds. IHC staining also depicted the appearance of smooth muscle, fibroblast, cardiomyocyte, and endothelial progenitor cells in the scaffolds after cells seeding process (FIGS. 7A-7E). An alternative route was proposed involving the decellularization of coronary-based right cardiac flap which is thinner than that of the left heart, with its attached pedicle and coroner. By doing this, the innate blood vessel infrastructure for upcoming revascularization was maintained. The ECM is obtained by cutting the whole native tissue with its inherent vasculature, before the decellularization process, and performs the perfusing with SDS via coronary artery, which facilitate the perfusion process.

The favorable outcomes of the present Example can be clarified as consequences of the outstanding effects induced by bioengineered ovine heart scaffold and applied GFP fetal cardiac cells. In the present Example, it was reported that the flourishing perfusion-based decellularization method of coronary-based right heart flaps for the development of a 3D scaffold with an intact microstructural architecture, with satisfactory recellularization results using neonatal cardiac cells of GFP rat. Choosing proper tissues mimicking the physiological structures of human tissues and finding the best decellularization method are vital steps for the expansion of biological scaffolds (Hussey G S, Dziki J L, Badylak S F. Extracellular matrix-based materials for regenerative medicine. Nat Rev Mater. 2018; 3(7):159-73). Hence, the concentration of the used detergent as well as the employed technique should be taken into consideration (Gilpin A, Yang Y, Decellularization strategies for regenerative medicine: from processing techniques to applications. Biomed Research International. 2017). The correlation between the percentage of residual DNA and adverse consequences after implantation is significant. As well, less than 50 ng/mg dry weight of the tissue is required for achieving an effective decellularization technique (Bruyneel A A, Carr C A. Ambiguity in the presentation of decellularized tissue composition: the need for standardized approaches. Artif Organs. 2017; 41(8):778-84). In the present Example, the decellularization process eliminated more than 95% of DNA contents and residual DNA was less than 50 ng/mg dry weight of decellularized tissue. Perfusion-based decellularization method of a whole organ is a proper but complex method for the whole organ decellularization, as it needs to remove all cellular components and deliver all the related detergents to the organ microstructure (The LE. Retraction-Engineered whole organs and complex tissues. Lancet. 2018; 392(10141):11). In the present Example, vascular networks were employed by using this efficient decellularization method and increasing the exposure of inner part of the heart tissue with detergents. The vascular network was preserved for further anastomosis during the transplantation (procedure. According to the study of Taylor et al., whole heart was decellularized using a perfusion-based technique, which resulted in construction of scaffolds with preserved ECM and functional vascular network (Taylor D. Sampaio L, Gobin A. Building new hearts: a review of trends in cardiac tissue engineering. Am J Transplant. 2014; 14(11):2448-59). In previous study, preparation and characterization of human size whole heart was well introduced for organ engineering, using scaffold microangiographic imaging. The successful decellularization of an entire ovine heart was also depicted for further development of a 3D scaffold with preserved microstructural construction (Akbarzadeh A, Khorramirouz R, Ghorbani F, Beigi R S H, Hashemi J, Kajbafzadeh A-M. Preparation and characterization of human size whole heart for organ engineering: scaffold microangiographic imaging. Reg Med. 2019; 14(10):939-54). In accordance with previous outcomes, ovine hearts were decellularized and assessed through coronary perfusion with acceptable results. Furthermore, according to the results of SEM examination and analysis of mechanical properties, the decellularized tissues maintained dense collagen networks. Here, concerns existed as to testing heart flap substitutes using sheep model, due to the similarities in the calcification and pathophysiology of degeneration between sheep and humans (Meuris B, Ozaki S, Herijgers P, Verbeken E, Flameng W, editors. Influence of species, environmental factors, and tissue cellularity on calcification of porcine aortic wall tissue. Semin Thorac Cardiovasc surg; 2001). Particularity, several cell types such as neonatal cardiomyocytes, mesenchymal stem cells (MSCs), induced pluripotent stem cells (iPSCs), and peripheral blood mononuclear cells (PBMNCs) have been used (Di Franco S, Amarelli C, Montalto A, Loforte A. Musumeci F. Biomaterials and heart recovery: cardiac repair, regeneration and healing in the MCS era: a state of the “heart”. J Thorac Dis. 2018; 10(Suppl 20):S2346; Hülsmann J, Aubin H, Wehrmann A, Lichtenberg A, Akhyari P. The impact of left ventricular stretching in model cultivations with neonatal cardiomyocytes in a whole-heart bioreactor. Biotechnol Bioeng. 2017; 114(5):1107-17). In the study of Porrello et al. cardiac regeneration was introduced in the newborn mouse due to previous findings that neonatal cardiomyocytes are still highly proliferative at that stage (Porrello E R, Mahmoud A I, Simpson E, Hill J A, Richardson J A, Olson E N et al. Transient regenerative potential of the neonatal mouse heart. Sci. 2011; 331(6020):1078-80). It has been also depicted that heart muscle is regenerated with proliferative capacity of neonatal cardiomyocytes after injury with robust angiogenesis and negligible fibrosis (Li J, Yang K Y, Tam R C Y, Chan V W, Lan H Y, Hori S et al. Regulatory T-cells regulate neonatal heart regeneration by potentiating cardiomyocyte proliferation in a paracrine manner. Theranostics. 2019; 9(15):4324). In the present Example, neonatal cardiac cells of GFP positive rats were used due to their proliferative effects with satisfactory in-vitro recellularization results. Ex-vivo perfusion of whole decellularized heart tissue could create a scaffold similar in architecture and cell populations. Stem cell-seeded biocompatible cardiac patches can be considered as an innovative therapeutic technique in order to augment cell transfer efficiency or survival, prevent LV remodeling, and prevent the rupture of aneurysm following Ml. As an implanted cardiac patch is under constant contraction, it should be strong and elastic enough to oppose damage from the contracting myocardium. As a result, providing vascular or cardiac patches may be more reachable and for all purposes achieving suitable ECM is fundamental. Cardiac patches used in this study were extremely elastic and flexible in mechanical investigations and seems to be suitable in the mechanically dynamic environment of the heart after implantation. However, the obtained results of the present in-vitro study showed that seeding of coronary-based right heart flap neonatal GFP rat cardiac cells may have the capability to result in satisfactory recellularization after the implantation. Pre-seeded decellularized aortic valve conduit with bone marrow-derived MSCs revealed promising functional potentiality and satisfactory results in postoperative cell seeding capacities (Kajbafzadeh A-M, Tafti S H A, Mokhber-Dezfooli M-R. Khorramirouz R, Sabetkish S. Sabetkish N et al. Aortic valve conduit implantation in the descending thoracic aorta in a sheep model: The outcomes of pre-seeded scaffold. Int J Surg. 2016; 28:97-105). Recent studies have offered thin myocardial-like constructs as implants, the results of which demonstrated improvements in myocardial function on transplantation in small animal models with myocardial infarction (MI) (Zimmermann W-H, Melnychenko I, Wasmeier G, Didié M, Naito H, Nixdorff U et al. Engineered heart tissue grafts improve systolic and diastolic function in infarcted rat hearts. Nat Med. 2006; 12(4):452-8). In one study in 2005, stents were used in patients with coronary artery aneurysms. The results showed that 5 of the 24 patients developed restenosis on follow-up angiography with aneurysms more than 10 mm in diameter (Szalat A, Durst R, Cohen A, Lotan C. Use of polytetrafluoroethylene-covered stent for treatment of coronary artery aneurysm. Catheter Cardiovasc Interv. 2005; 66(2):203-8). In one study in 2011, it has been shown that the application of thin decellularized hearth slices as patch-based therapies, provide limited regeneration capacities in animal models (Sarig U. Machluf M. Engineering cell platforms for myocardial regeneration. Exp Opin Biol Ther. 2011; 11(8):1055-77). Previous studies have verified the poor mechanical properties and unfortunate cell seeded process using synthetic biodegradable scaffold after implantation (Sodian R, Hoerstrup S P, Sperling J S, Daebritz S, Martin D P, Moran A M et al. Early in vivo experience with tissue-engineered trileaflet heart valves. Circ. 2000; 102(suppl_3):Iii-22-Iii-9). The results of the current study demonstrated the efficacy of cardiac patches engineered with GFP rat neonatal cardiac cells in superior elastic mechanical properties which may promote appropriate cellular interaction after implantation. The pre-seeded scaffolds described in the present Example can be a possible alternative for regeneration of individual cardiac components such as cardiac wall aneurysm after a MI or as a ventricle for congenital heart disease, with some unique advantages over current medical therapies. This study provides a supportive basis for cardiovascular engineering by grafting or replacing the infarcted part of the heart with the engineered functional patch by CABG or implanting biological cardiac patch in ventricular aneurysm surface. The outcomes of the application of these pre-seeded scaffolds to prevent left ventricle aneurysm from rupture following MI, as well as the assessment of growth factor enrichment in implanted decellularized tissues will be evaluated for potential clinical application in future studies.

The experiments show that a heart ECM seeded with GFP⁺ rat neonatal cardiac cells can be used as a patch for prevention of an aneurysm rupture developed after MI. The examples indicate that this pre-seeded scaffold is an appropriate surgical procedure to foster cardiac tissue repair.

1-32. (canceled)

33. A method of producing a decellularized mammalian cardiac tissue matrix comprising:
a) cannulating a mammalian right heart flap, mammalian right heart, or mammalian right heart artery,
b) perfusing said right heart flap or right heart with distilled water,
c) perfusing said right heart flap or right heart with 1% SDS, and
d) rising said right heart flap or right heart with phosphate buffered saline (PBS), containing 100 U/ml penicillin, 100 U/ml streptomycin,
thereby producing a decellularized cardiac tissue matrix.

34. The method of claim 33, wherein all solutions comprise antimycotic solutions and 1% (v/v) antibiotic or wherein all steps are performed under sterile conditions.

35. (canceled)

36. A method of producing recellularized tissue or recellularized organ by repopulating a decellularized tissue matrix or a decellularized organ matrix with cells.

37. The method of claim 36, wherein said decellularized tissue matrix or decellularized organ matrix is produced by the methods of claim 33.

38. The method of claim 36, wherein said decellularized tissue matrix or decellularized organ matrix is produced by perfusion-based methods, is a perfusion-based decellularized heart flap, is a perfusion-based decellularized right heart flap, or is produced by immersion in a decellularization solution.

39. (canceled)

40. (canceled)

41. (canceled)

42. The method of claim 36, wherein said repopulating comprises seeding the decellularized tissue matrix or decellularized organ matrix with cells.

43. The method of claim 36, wherein said cells are mammalian cardiac cells.

44. The method of claim 36, wherein said cells are neonatal green fluorescent protein (GFP) positive cardiac cells.

45. The method of claim 36, wherein said cells are rats' neonatal green fluorescent protein (GFP) positive cardiac cells.

46. The method of claim 36, wherein said cells are GFP positive rat neonatal cardiac cells.

47. The method of claim 36, wherein said cells are stored in Dulbecco's Modified Eagle Medium (DMEM) with about 10% fetal bovine serum (FBS) at about 37° C. in a CO2 incubator.

48. (canceled)

49. The method of claim 36, wherein said method is performed ex vivo.

50. The method of claim 36, wherein said method is performed in vitro.

51. The method of claim 36, wherein said recellularized organ is a recellularized heart.

52. The method of claim 36, wherein said recellularized tissue is recellularized cardiac tissue.

53. The method of claim 52, wherein said recellularized cardiac tissue is a recellularized cardiac tissue scaffold.

54. The method of claim 52, wherein said recellularized cardiac tissue is a recellularized cardiac patch or recellularized cardiac graft.

55. The method of claim 52, wherein said recellularized cardiac tissue or recellularized heart is a recellularized right heart flap or recellularized right heart.

56. The method of claim 52, wherein said recellularized cardiac tissue is a recellularized heart ECM.

57. A method of producing a recellularized cardiac flap by repopulating a decellularized cardiac flaps with GFP positive rat neonatal cardiac cells, the method comprising:
a) connecting the decellularized cardiac flaps to a closed-system bioreactor that is placed in a cell culture incubator with about 5% CO2 at about 37° C.,
b) circulating the medium with 10 mL/min via the right coronary of said cardiac flap for about 24 h,
c) removing the decellularized cardiac flaps,
d) injecting the flaps with ten million GFP+ rat neonatal cardiac cells via the right coronary of the cardiac flaps,
e) injecting 5 million GFP+ rat neonatal cardiac cells into five different areas of the myocardium 5 days after cell injection through right coronary artery,
f) injecting another 5 million GFP+ rat neonatal cardiac cells into five different areas of the myocardium 10 days after cell injection through right coronary artery,
thereby producing the recellularized cardiac flap.

58-89. (canceled)
